(19) United States
(12) Patent Application Publication
(10) Pub. No.: US 2014/0141405 A1
(43) Pub. Date: May 22, 2014

(54) Method for Increasing the Biomass and the Metabolic Activity of Microorganisms by the Combined Adjustment of the Oxidation-Reduction Potential and of the Oxygen Dissolved During the Fermentation Process

(71) Applicant: L'Air Liquide, Societe Anonyme pour I'Etude et Exploitation des Procedes Georges Claude, Paris (FR)

(72) Inventors: Armelle Marecat, Massy (FR); Dominique Ibarra, Gif-Sur-Yvette (FR); Isabelle Auzanneau, Dange Saint Romain (FR); Jean-Roch Mouret, Dange Saint Romain (FR); Jean-Philippe Obert, Dange Saint Romain (FR); Loic Damongeot, Valenton (FR); Olivier Roy, Issy Les Moulineaux (FR)

(73) Assignee: L'Air Liquide, Societe Anonyme pour I'Etude et Exploitation des Procedes Georges Claude, Paris (FR)

(21) Appl. No.: 14/165,027

(22) Filed: Jan. 27, 2014

Related U.S. Application Data
(63) Continuation of application No. 12/867,377, filed on Jan. 5, 2011.

(30) Foreign Application Priority Data
Feb. 15, 2008 (FR) 0850976

(51) Int. Cl. C12Q 3/00

(52) U.S. Cl. CPC C12Q 3/00
USPC 435/3

(57) ABSTRACT

The invention relates to a method for cultivating microorganisms, particularly of the type that comprises the step of seeding a culture medium with one or more microorganism strains, and the step of cultivating the medium thus seeded, characterized in that it comprises, during the entirety or a portion of the cultivation, the two following and simultaneous adjustments: adjusting the amount of oxygen dissolved in the medium to a given dissolved-oxygen setpoint; adjusting the value of the redox potential Eh of the medium to a given setpoint value Eh.

### CROSS-REFERENCE TO RELATED APPLICATIONS

This application is a continuation of U.S. patent application Ser. No. 12/867,377, filed Jan. 5, 2011, which is a §371 of International PCT Application PCT/FR2009/050174, filed Feb. 5, 2009, which claims §119(a) foreign priority to French application 0850976, filed 15 Feb. 2008, the entire contents of which are incorporated herein by reference.

### BACKGROUND

1. Field of the Invention

The present invention relates to a method for cultivating microorganisms in order to increase the biomass and/or the metabolic activity of microorganisms (i.e. to increase the production of certain metabolites, namely certain molecules produced by the microorganisms), this by the combined adjustment of the oxidation-reduction potential and the dissolved oxygen.

As an illustration, the invention may relate for example to lactic bacteria, but also to microorganisms having preferably an anaerobic metabolism such as yeasts.

The invention also relates to the field of certain finished products, such as wine or beer, for which the production method employs a fermentation step.

The actual cultivation step takes place in a vessel, with or without stirring, in a culture medium of which the composition is suited to the specific requirements of each microorganism. The composition of the culture medium may be extremely varied, but mention is usually made of the presence of one or more elements among polysaccharides, glycerol, milk, glucose, etc.

Similarly, parameters such as for example pH, temperature and dissolved oxygen pressure may be adjusted.

2. Related Art

Various types of cultivation procedure exist:
a discontinuous or “batch” culture, used notably for the production of lactic ferments or bread-making yeast,
a semi-continuous of “fed-batch” culture, used for example for the production of ferments sensitive to a fermentation product or for the production of a biomass sensitive to inhibition by the fermentation substrate,
a continuous culture with or without recycling, the latter being used notably for the production of ferments, molecules of interest, and for the biological purification of wastewater.

The step of preserving the biomass may be carried out in liquid form, by freezing, by cryopreservation, by freeze drying or by drying. Protective agents are used to protect the microorganisms from the harmful effects of preservation treatments.

Freeze drying is known to be a low temperature dehydration operation, which consists of removing the major part of the water contained in the product, after freezing, by sublimation.

As has been said, microorganisms are widely used in the food field, notably lactic bacteria, such as probiotic cultures or ferments. The viability and metabolic activity of these lactic bacteria, produced on a large scale for their criteria of technological ability, may be affected by the various steps that they undergo during their production (inoculation, cultivation and concentration) and their preservation (freezing or freeze drying).

Microorganisms may also be made use of in the production of biofuels, notably by the conversion of sugar into ethanol. Microorganisms also enable molecules of interest to be produced, notably in the pharmaceutical field.

Optimization of the biomass and/or of the metabolic activity of these cultures is therefore very important and of necessity has both technological and economic importance.

Environmental parameters play a key role in the growth of microorganisms, in the metabolic reactions and in the physiological mechanisms responsible for the activity of microorganisms.

Like the pH, temperature or composition of the medium, the value of the oxidation-reduction potential seems to have an effect on the growth and viability of bacterial strains. In point of fact, several studies on lactic bacteria have demonstrated an effect of the oxidation-reduction potential on metabolic flow, the survival of probiotic ferments and the production and/or stability of molecules of interest. In this respect, reference may be made to the following documents: documents EP-1 856 241 and EP-1 649 755 as well as WO 2007/036693 A1 in the name of the Applicant, or patent U.S. Pat. No. 7 078 201.

It will be noted that document WO 2007/036653 relates to the adjustment of the redox potential at one or more steps of a method for producing a food or biotechnological product comprising a fermentation step, so as to perform at least one of the steps of the method under reducing conditions and at least one of the steps of the method under oxidizing conditions, and notably making it possible to alternate the phases of the fermentation considered under reducing conditions with the phases of the fermentation considered under oxidizing conditions.

Various compounds may be used as a reducing agent and for inducing a reduction in and/or maintenance of the oxidation-reduction potential. Among these, mention may be made of sulfites and ammonia but also of reducing gases such as hydrogen or mixtures containing hydrogen.

These studies of the prior art have made it possible to demonstrate that the use of reduced media, media where the oxidation-reduction potential is low, makes it possible to increase the biomass produced but also the viability of the product during its subsequent preservation, and also makes it possible to increase the production of metabolites.

Another means reported by the prior art for increasing the biomass produced is the use of a small quantity of oxygen. Although oxygen has an inhibiting effect on lactic bacteria, facultative anaerobic bacteria, it is described in the literature as being able to be of benefit to their growth. Thus, studies have shown that for an MG1363 strain of Lactococcus lactis, addition of oxygen enables the biomass produced to be increased. In this way, the concentration of biomass moves from 0.54 g/l (dry weight) in an anaerobic culture to 0.68 g/l for a culture to which a percentage of dissolved oxygen of 5% is added (reference will be made to the studies by Jensen et al. entitled “Metabolic behavior of Lactococcus lactis MG1363 in microaerobic continuous cultivation at a low dilution rate” that appeared in AEM—2001, Vol. 67, No. 6).

It would then appear that the two solutions recommended by the prior art, namely the establishment of a low redox potential in the first case and action on the quantity of dissolved oxygen in the second case, clearly seem to be incompatible. Indeed, since oxygen is a powerful oxidizing agent, adding it to the culture medium will significantly increase the redox potential.

### SUMMARY OF THE INVENTION

As will be seen below in greater detail, it is therefore the merit of the present invention to have demonstrated that it is possible to provide and adjust a quantity of dissolved oxygen while adjusting the redox potential to a desired fixed low value, this bringing about, in an extremely advantageous manner, a significant increase in the productivity of cultivated strains, while action on the two aforementioned parameters could appear to a person skilled in the art at first sight, in view of the prior art referred to above and highly logically, to be destructive.

The present invention thus provides a method employing simultaneous adjustments, preferably using gases or gaseous mixtures to ensure these adjustments:
advantageously, adjustment of the redox potential will use injection of a reducing gas or gaseous mixture (for example a gaseous mixture containing hydrogen) into the culture medium. It is considered that the addition of chemicals such as ascorbic acid (vitamin C) for example could also be employed to bring about this adjustment of the redox, but as has been said, it will be preferred to use the injection of suitable gases,
while, advantageously, adjustment of the dissolved oxygen value will be made by injecting air or pure oxygen or oxygen-enriched air into the culture medium or any gas capable of releasing oxygen, for example mixtures containing oxygen and CO₂, etc.
these adjustments are carried out during the entirety or part of the fermentation, namely during the entirety or part of the cultivation of the bacterial strain.

It will be noted that document U.S. Pat. No. 7,078,201 referred to above indicates that an optimum value of the redox potential may increase the production of ethanol, reduce the formation of glycerol and reduce the fermentation time as compared with a conventional fermentation. The method proposed by this document proposes for this the maintenance of a value of the redox potential in the fermenter of between −250 and +50 mV by means of continuous aeration of the medium by air injection. The objective sought by the authors of the document is to proceed counter to a lowering of the redox potential, and for this, according to one of the methods recommended by the document, the author recommends adding sodium hydroxide in order to limit the reduction of the value of the redox potential instead and in place of the traditional use of ammonia (considered as too reducing) in order to adjust the pH during growth.

It will therefore be understood that the authors do not at any time refer to stable and continuous adjustment of the redox to a fixed value being made (fine adjustment) and even less do they refer to the fact of adjusting the quantity of dissolved oxygen while adjusting the redox potential to a desired fixed low value (the dissolved oxygen content which would naturally have the tendency to raise the redox potential).

The present invention then relates to a method for cultivating microorganisms, a method of the type where, in particular, a step is performed of seeding a culture medium with one or more microorganism strains, and a step of cultivating the medium seeded in this way, which is characterized in that, during the entirety or part of the cultivation, two of the following adjustments are made simultaneously:
the quantity of dissolved oxygen in the medium is adjusted to a given dissolved oxygen set point,
the value of the redox potential Eh of the medium is adjusted to a given set point value for Eh.

It should be understood on reading the preceding account that according to the invention, the value of the redox potential Eh of the medium is adjusted to a set point value that is less than the value that would be naturally reached by only adjusting the dissolved oxygen.

The invention could moreover adopt one or more of the following technical features:
the value of the redox potential adjusted in this way is negative;
the redox potential is adjusted within the range extending from −400 to 0 mV;
the dissolved oxygen value is adjusted within the range extending from 1 to 30%;
different adjustment sequences are performed according to the growth phase concerned, by employing different couples (set point value of the redox potential, dissolved oxygen set point) according to the phase of the growth of said strain considered;
adjustment of the redox potential uses the injection of a reducing gas or gaseous mixture into the culture medium, such as a gaseous mixture containing hydrogen, while adjustment of the dissolved oxygen value uses an injection of air or of a gas or gaseous mixture capable of releasing oxygen such as pure oxygen or oxygen-enriched air into the culture medium;
adjustment of the redox potential uses the injection of a chemical compound into the culture medium;
the two simultaneous adjustments are made by an adjustment of the PID type according to the following procedure:
   an evaluation is first of all made of the impact of the injection of air or of oxygen or of a mixture able to release oxygen, and of a reducing gas or gaseous mixture, on the one hand, on the dissolved oxygen value and, on the other hand, on the redox potential, by means of performing preliminary tests employing variable gas flows injected into the medium;
   two flow rate controllers are used, capable of adjusting the flow rate of the gas able to release oxygen and the flow rate of the reducing gas or gaseous mixture into the culture medium. These controllers constitute actuators of the regulating system;
   a control system is used with data acquisition, for example an automaton that periodically examines each of the flow rate controllers, as regards its set point value and the value of the measurement of the parameter with which it is associated, and which consequently corrects its output;
   the corrective measures by each controller were determined from said impact evaluations as a function of the disturbance of the system for given set points for the redox potential and dissolved oxygen.

### BRIEF DESCRIPTION OF THE FIGURES

FIG. 1 illustrates in a schematic manner the experimental assembly used for carrying out the tests.

FIG. 2 is a graph of redox potential and dissolved oxygen versus time.

### DETAILED DESCRIPTION OF THE INVENTION

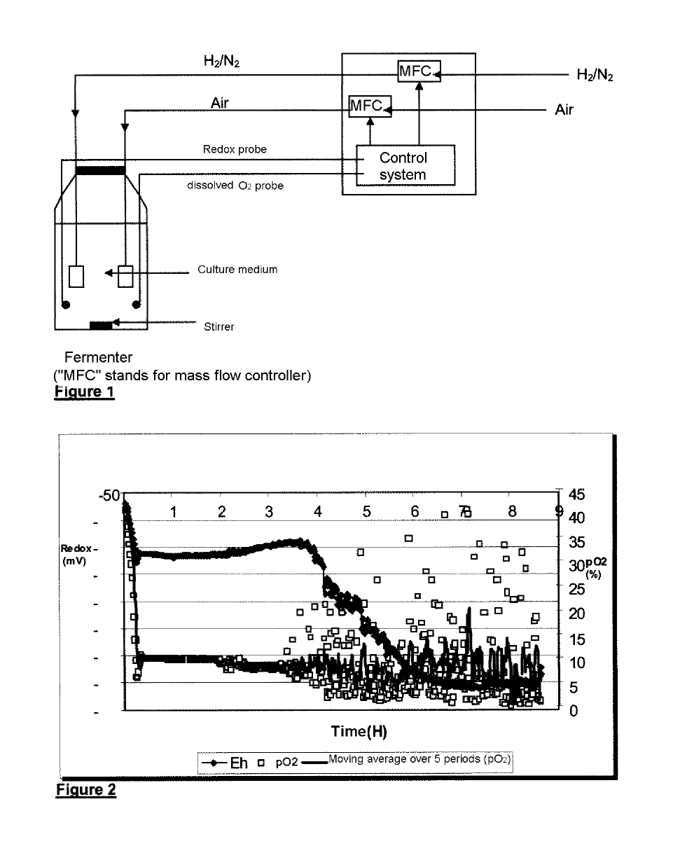

The invention will be better understood on reading the following example with reference to FIG. 1 that illustrates in a schematic manner the experimental assembly used for carrying out the tests.

Tests were carried out on a mesophilic strain, Lactococcus lactis, in a fermenter with a capacity of 1.5 liters.

The fixed target values (set points) for the oxidation-reduction potential and the dissolved oxygen were respectively −200 mV and 10%.

Here is what should be understood by this 10% dissolved oxygen value: the apparatus used is capable of measuring the oxygen concentration of the culture medium by means of a probe. This probe is calibrated in the following way: an H₂/N₂ mixture (4/96) is injected into the culture medium in order to draw off all the oxygen present. At this stage, the probe should then indicate a concentration of 0% dissolved oxygen. Secondly, the aforementioned H₂/N₂ mixture is replaced by air, which is injected in a large quantity (typically at the maximum flow rate that the system can provide). After waiting until the measured value is stable, the system should then indicate a value of 100% dissolved oxygen. If this is not the case, the probe is calibrated with a value of 100% being given to the probe. This “100% dissolved oxygen” then corresponds to the maximum oxygen that the medium considered can dissolve. Once this calibration has been carried out, adjustment of dissolved oxygen can be activated with the set point that that it is desired to establish in the culture medium (here for example 10%). Tests were then carried out with 10% of the maximum that the medium considered could dissolve.

Similarly, one embodiment of the two simultaneous adjustments according to the present invention will be explained in detail hereinafter.

An automaton is used that periodically (according to a period that has been imposed on it, for example less than a second), examines each of the mass flow controllers, namely its set point value and the value of the measurement of the parameter considered and that consequently corrects its output, i.e. the instruction, which it gives as feedback. In this case, use is made of a control of the PID type which here makes it possible to adjust both the redox and the dissolved oxygen. As will be explained in greater detail hereinafter, it should in point of fact be pointed out that the redox potential and the dissolved oxygen do not have the same behavior, and they do not have the same time constants or the same reaction amplitude for an identical flow rate variation. Consequently, the parameters of each controller are not identical and fine adjustment employed on each PID enables the controllers not to oscillate, this implementation thus limiting interferences between them.

The implementation described above is only an illustration of one embodiment, which does not of course exclude other means of adjustment and this without at any time departing from the scope of the present invention.

The inputs and outputs of the controller typically installed are described below:

### For the Redox:

As input
i) A redox set point in millivolts given from the man-machine interface (MMI)
j) Measurement of the redox in millivolts conveyed by the sensor immersed in the culture medium

As output:
k) Flow rate of H₂/N₂ conveyed into the medium.

### For Dissolved Oxygen

As input
i) Dissolved O₂ set point expressed as a percentage (0-100%) and that was entered from the man-machine interface (MMI)
j) Measurement of dissolved O₂ conveyed by the corresponding probe in mV and converted into a percentage so as to have the same unit as that of the set point.

As output
k) Flow rate of air conveyed to the medium

More precisely, as an example of the implementation indicated above:
a controller of the PID type is used that is in fact a proportional-integral controller. Other types of controller could also be used, as for example an internal model or fuzzy logic controller,
as has been indicated above, the redox potential and dissolved oxygen do not have the same behavior and they do not have the same time constants or the same amplitude for an identical flow rate variation. Consequently, the parameters for each controller are not identical and the suitable adjustment employed on each PID makes it possible for the controllers not to oscillate, which in this way limits interferences between them. More precisely, what makes it possible according to the invention to differentiate between parameters of each controller, is the previous quantification of the impact of air or other gas capable of releasing oxygen and of the mixture containing hydrogen on the dissolved oxygen value on the one hand and on the redox potential on the other hand. This quantification was made by carrying out preliminary tests where variations were established of the gas flows injected into the medium. These tests made it possible to determine the impact of air injection on the redox potential and on the dissolved oxygen, and then the impact of injecting a hydrogen-containing mixture on the redox potential and on dissolved oxygen. These tests made it possible to know the impact of each gas on its measured reference quantity but also on its impact (or disturbance that it produces) on the other quantity measured. These experiments made it possible to conclude that in the case studied (gaseous mixtures, medium treated, etc.):
the influence of the hydrogen-containing mixture on dissolved oxygen is approximately four times less than that of air,
as regards the redox potential, air has an impact 10 times less than that of the hydrogen-containing mixture.

Taking account of this identification of transfer functions, it is thus possible to determine the parameters of each corrector, the objective being that the system reacts correctly to a disturbance of the system for a fixed point. In point of fact, according to the invention, a given set point is established during all or part of the growth, and on the other hand the effect of the growth of bacteria in the culture medium should be included in the determination of corrector parameters. It is known in point of fact that the growth of bacteria has an influence on the redox potential: the redox potential falls during growth and oxygen consumption increases. It consists of a phenomenon that disturbs adjustments: adjustment of the correctors is made to react to this disturbance as best as possible.

In this way, it is the combination of identifications made and the manner in which the corrector is adjusted (response to a disturbance and not to a change of set point) that makes it possible to obtain the results observed with the use of “simple” controllers of the PID (monovariable) type.

The objective of the adjustment during the tests referred to above is to maintain, during at least part of the growth of the strain, a constant value of the redox potential as well as a constant dissolved oxygen value (this will indeed be seen moreover in FIG. 2 that will be commented on hereinafter).

The biomass, the acidifying activity and the productivity per operation were measured at the end of fermentation (end of cultivation) but also on frozen pellets and on the freeze-dried product. These results were compared with a control culture in which neither the redox potential nor the dissolved oxygen were adjusted (an aerobic culture prepared with a simple flushing with nitrogen at 0.5 l/min in the headspace of the fermenter).

The results shown in FIG. 2 show tracking of the adjustment of dissolved oxygen values (pO₂) and of the oxidation-reduction potential (Eh) for a strain of Lactococcus lactis.

The various gains obtained during various production steps are described in the table below.

The essential result of this invention is demonstrated, according to which it is possible, by a system of controlled adjustment, to uncouple the two parameters while adjusting them simultaneously: oxidation-reduction potential and dissolved oxygen.

It is then found that the gains obtained are greater than 50% at the step of the freeze-dried product, which is very satisfying. By simultaneously adjusting the two parameters, oxidation-reduction potential and dissolved oxygen, large gains are obtained in biomass and acidifying activity.

Table of results obtained

| | End of | | Freeze-dried |
|---|---|---|---|
| | fermentation | Frozen pellets | product |
| Gain in biomass | 28% | 45% | 55% |
| (in %) | | | |
| Gain in acidifying | 17% | 19% | 71% |
| activity (in %) | | | |
| Gain in | 17% | Not determined | 71% |
| productivity (in | | | |
| %) | | | |

As it will appear clearly to a person skilled in the art, the optimum dissolved oxygen and redox potential values will have to be adapted according to the strains and targeted objective (production of biomass and/or production of metabolites).

The performance of different adjustment sequences may also be envisaged as a function of the growth phase concerned, that is to say the establishment of different couples (value of redox/pO₂) as a function of the growth phase of the strain, and a numerical example is given below as an illustration:
at the start of growth, a low redox value close to −400 mV and a dissolved oxygen value close to 10%,
then, in an exponential phase, a redox value close to −400 mV and a dissolved oxygen value close to 5%,
and in a stationary phase, a redox value close to −400 mV and an oxygen value close to 0%.

It will be understood that many additional changes in the details, materials, steps and arrangement of parts, which have been herein described in order to explain the nature of the invention, may be made by those skilled in the art within the principle and scope of the invention as expressed in the appended claims. Thus, the present invention is not intended to be limited to the specific embodiments in the examples given above.

What is claimed is:

1. A method for cultivating microorganisms comprising the steps of:
a) seeding a culture medium with one or more microorganism strains,
b) cultivating the seeded medium, and
c) during at least part of the cultivation, simultaneously adjusting:
i) a quantity of dissolved oxygen in the medium to a selected dissolved oxygen set point, and
ii) a value of the redox potential (Eh) of the medium to a selected Eh set point value.

2. The cultivation method of claim 1, wherein the selected Eh set point value is negative.

3. The cultivation method of claim 1, wherein the selected Eh set point value is from −400 to 0 mV.

4. The cultivation method of claim 1, wherein the dissolved oxygen set point is from 1 to 30%.

5. The cultivation method of claim 1, wherein step c) comprises a different combination of the selected dissolved oxygen set point and the selected Eh set point value for two or more growth phases of said strain.

6. The cultivation method of claim 1, wherein sub-step c) i) comprises a step of injecting a reducing gas or reducing gaseous mixture into the culture medium and wherein sub-step c) ii) comprises injecting air or injecting a gas or gaseous mixture capable of releasing oxygen.

7. The method of claim 6 wherein the reducing gas comprises hydrogen and/or wherein the gas or gaseous mixture capable of releasing oxygen is pure oxygen or oxygen-enriched air.

8. The cultivation method of claim 1, wherein step c) ii) comprises injecting into the culture medium a compound capable of causing a change in Eh in the medium.

9. The cultivation method of claim 1, wherein the two simultaneous adjustments c) i) and c) ii) are executed by a proportional-integral-derivative controller (PID controller) operably connected to
a) at least two flow rate controllers wherein one flow rate controller capable of adjusting a flow rate of a gas able to release oxygen and the second flow rate controlled capable of adjusting the flow rate of a reducing gas or reducing gaseous mixture into the culture medium,
b) a sensor capable of measuring the dissolved oxygen in the medium, and
c) a sensor capable of measuring the redox potential (Eh) of the medium.

10. The method of claim 9, wherein the relation of the flow rates of each gas to both the dissolved oxygen value and the redox potential of the culture medium are measured in preliminary tests employing variable gas flows injected into the medium.

11. The method of claim 10 wherein the PID controller is capable of modifying the flow rates in response to data from the sensors to readjust the dissolved oxygen value and/or the redox potential of the culture medium by correlating the sensor data and the flow rates with the data set from the preliminary tests.
